(19) United States
(12) Patent Application Publication
(10) Pub. No.: US 2004/0001796 A9
(43) Pub. Date: Jan. 1, 2004

(54) Cosmetic compositions containing a methacrylic acid copolymer, a dimethicone, a nacreous agent and a cationic polymer, and uses thereof

(76) Inventor: Mireille Maubru, Chatou (FR)

Correspondence Address: Thomas L. Irving, FINNEGAN, HENDERSON, FARABOW, GARRETT & DUNNER, L.L.P., 1300 I Street, N.W., Washington, DC, 20005-3315, US

(21) Appl. No.: 10/237,785

(22) Filed: Sep. 10, 2002

(30) Foreign Application Priority Data
Sep. 11, 2001 (FR) 01 11745

(51) Int. Cl. A61K 7/06; A61K 7/11

(52) U.S. Cl. 424/70.12; 424/70.16

(57) ABSTRACT

Cosmetic compositions comprising, in a cosmetically acceptable medium, at least one copolymer of methacrylic acid/C₁-C₄ alkyl acrylate, at least one polymer chosen from cationic and amphoteric polymers whose cationic charge density is less than 1 meq/g, at least one nacreous and/or opacifying agent and at least one silicone chosen from polydialkylsiloxanes comprising trimethylsilyl end groups. These compositions can be used for washing and/or conditioning keratin materials such as the hair or the skin.

The present invention relates to novel cosmetic compositions comprising, in a cosmetically acceptable medium, at least one methacrylic acid/C₁-C₄ alkyl acrylate copolymer, at least one particular polymer chosen from cationic and amphoteric polymers, at least one agent chosen from nacreous and opacifying agents and at least one particular silicone.

Nacreous and/or opacifying agents are commonly used in cosmetic compositions, for example, detergent compositions such as shampoos, in order to give these shampoos a nacreous appearance, which is preferred by consumers. It has been found that these nacreous agents do not give the hair satisfactory conditioning properties.

There is thus a need for a detergent cosmetic composition, such as a shampoo, which can have a nacreous and/or opacified appearance while at the same time giving acceptable cosmetic performance qualities on keratin materials, such as the hair and the scalp, and, for example, as regards the lightness, softness and feel of the hair.

It is well known that hair, which has been sensitized (i.e. damaged and/or embrittled) to varying degrees under the action of atmospheric agents or under the action of mechanical or chemical treatments, such as dyes, bleaches and/or permanent-waving, may often be difficult to disentangle and to style, and may lack softness.

It has already been recommended to use conditioners, such as cationic polymers or silicones, in compositions for washing or caring for keratinous material such as the hair, in order to be able to disentangle the hair and to give it softness and flexibility. However, at least one of the cosmetic advantages mentioned above is also unfortunately accompanied, on dried hair, by certain cosmetic effects considered as being undesirable, i.e. lankness of the hairstyle (lack of lightness of the hair) and lack of smoothness (hair not uniform from the root to the tip).

In addition, the use of cationic polymers for this purpose can have various drawbacks. On account of their high affinity for the hair, some of these polymers can become deposited thereon to a large extent during repeated use, and can lead to adverse effects such as an unpleasant, laden (charged or loaded) feel, stiffening of the hair and interfibre adhesion, which has an effect on styling. These drawbacks can be accentuated in the case of fine hair, which lacks liveliness and body.

Functionalized silicones are generally used in shampoo compositions as conditioners for improving the softness, feel and disentangling of the hair. However, it has been found that these silicones can lead to the formation of an unattractive layer at the surface of the shampoo, which can be harmful to the performance of the shampoo. To avoid the appearance of this phenomenon, stabilizers such as crosslinked acrylic polymers of the Carbopol type are frequently used. However, these stabilizers can have the drawback of reducing the cosmetic performance of shampoos, such as by making the hair more laden and coarser.

In summary, it is found that the current cosmetic compositions containing cationic or amphoteric polymers are not entirely satisfactory.

Certain cosmetic compositions, such as detergents, have been disclosed, containing a copolymer of methacrylic acid and of an alkyl acrylate, as a stabilizer or suspension agent for water-insoluble ingredients, for instance silicones or fatty substances. Such compositions have been described, for example, in patent application WO 01/76552. The foam quality and the cosmetic properties obtained with these compositions are still not sufficiently satisfactory.

The inventor has now discovered that the combination of at least one methacrylic acid/C₁-C₄ alkyl acrylate copolymer, at least one polymer chosen from cationic and amphoteric polymers whose cationic charge density is less than 1 meq/g, at least one agent chosen from nacreous and opacifying agents and at least one particular silicone having a particular particle size, makes it possible to overcome at least one of these drawbacks.

Specifically, it has been found that the use of the said acrylic copolymer in the compositions of the present invention can produce on keratin materials, such as the hair, good cosmetic properties, for example, as regards the lightness, softness, smooth feel, suppleness and manageability of dried hair. It has also been found that, with the compositions of the invention, dried hair that looks generally smoother can be obtained.

Moreover, the compositions according to the invention can be stable and can have an attractive visual appearance. The usual properties (appearance, consistency, foam abundance, elimination of foam) can be satisfactory.

The compositions of the invention, when applied to the skin, such as in the form of a bubble bath or a shower gel, can give an improvement in the softness of the skin.

Thus, according to the present invention, novel cosmetic compositions are now proposed, comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

Another aspect of the invention is also the composition as defined above, as well as methods, to give the hair sheen, lightness, softness, a smooth feel and suppleness.

Another aspect of the invention relates to a process for treating keratin materials, such as the hair, characterized in that it comprises applying to the keratin materials cosmetic compositions according to the invention.

According to the present invention, the expression “keratin materials” means the hair, the eyelashes, the eyebrows, the skin, the nails, mucous membranes or the scalp.

Another aspect of the invention relates to the addition of at least one copolymer chosen from crosslinked and non-crosslinked copolymers of methacrylic acid and of a C₁-C₄ alkyl acrylate in, or for the manufacture of, a cosmetic composition comprising at least one polymer chosen from cationic and amphoteric polymers whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

The various aspects of the invention will now be detailed. All the meanings and definitions of the compounds used in the present invention given below are valid for all the aspects of the invention.

One of the characteristics of the invention is the presence of at least one copolymer chosen from crosslinked and non-crosslinked copolymer of methacrylic acid and of a C₁-C₄ alkyl acrylate.

The methacrylic acid residue is, for example, in an amount ranging from 20% to 80% by weight and further, for example, from 25% to 70% by weight and even further, for example, from 35% to 60% by weight relative to the total weight of the copolymer.

The alkyl acrylate residue is, for example, in an amount ranging from 15% to 80% by weight and further, for example, from 25% to 75% by weight and even further, for example, from 40% to 65% by weight relative to the total weight of the copolymer. It is chosen, for example, from methyl acrylate, ethyl acrylate and butyl acrylate residues.

This copolymer is, for example, partially or totally crosslinked with at least one standard crosslinking agent. The crosslinking agents are, for example, polyunsaturated compounds, such as ethylenically polyunsaturated compounds. These compounds are, for example, chosen from polyalkenyl ethers of sucrose and of polyols, diallyl phthalates, divinylbenzene, allyl (meth)acrylate, ethylene glycol di(meth)acrylate, methylenebisacrylamide, trimethylolpropane tri(meth)acrylate, diallyl itaconate, diallyl fumarate, diallyl maleate, zinc (meth)acrylate, and derivatives of castor oil and of polyols manufactured from unsaturated carboxylic acids.

Crosslinking agents that may also be used include, for example, unsaturated monomer compounds comprising a reactive group capable of reacting with an unsaturation to form a crosslinked copolymer.

The content of the crosslinking agent generally ranges, for example, from 0.01% to 5% by weight and further, for example, from 0.03% to 3% by weight and even further, for example, from 0.05% to 1% by weight relative to the total weight of the copolymer.

According to one embodiment of the present invention, the copolymer of the invention may be, for example, in the form of a dispersion in water. The number-average size of the particles of copolymer in the dispersion is generally, for example, from 10 to 500 nm, as measured by appropriate means known to those skilled in the art, and further, for example, from 20 to 200 nm and even further, for example, from 50 to 150 nm.

These copolymers are described, for example, in patent application WO 01/76552.

Use can be made, for example, of the methacrylic acid/ethyl acrylate crosslinked copolymer in the form of an aqueous 30% dispersion manufactured and sold under the name Carbopol Aqua SF-1 by the company Noveon.

The copolymer concentration is generally from 0.01% to 10% by weight relative to the total weight of the composition and, for example, from 0.1% to 5% by weight relative to the total weight of the composition.

The at least one agent chosen from nacreous and opacifying agents of the invention may be chosen from:

i)esters of polyols comprising at least two carbon atoms and of long-chain fatty acids, for example, C₁₀-C₃₀ fatty acids and further, for example, C₁₆-C₂₂ fatty acids; these compounds may optionally be oxyethylenated.

Among these compounds, mention may be made, for example, of ethylene glycol monostearate and ethylene glycol distearate.

ii) long-chain fatty acid alkanolamides, for example, C₁₀-C₃₀ fatty acid alkanolamides and further, for example, C₁₆-C₂₂ fatty acid alkanolamides, such as stearic monoethanolamide, stearic diethanolamide, stearic monoisopropanolamide and stearic monoethanolamide stearate;

iii)esters of long-chain (e.g., C₁₀-C₃₀) monoalcohols and of long-chain (e.g., C₁₀-C₃₀) fatty acids, such as cetyl palmitate;

iv)long-chain fatty alkyl ethers such as C₁₀-C₃₀ fatty alkyl ethers that are solid at a temperature of less than or equal to approximately 30° C., such as the dialkyl ethers of formula (I):

R—O—R′ (I)

in which R and R′, which may be identical or different, are chosen from saturated and unsaturated, linear and branched alkyl radicals comprising from 10 to 30 carbon atoms and, for example, from 14 to 24 carbon atoms, R and R′ being chosen such that the compound of formula (I) is solid at a temperature of less than or equal to approximately 30° C. For example, R and R′ are each a stearyl radical. These compounds can be prepared, for example, according to the process described in patent application DE 41 27 230. One distearyl ether, which can be used in the context of the present invention, is sold under the name Cutina STE by the company Cognis;

v)long-chain (e.g., C₁₀-C₃₀) esters of long-chain (e.g., C₁₀-C₃₀) alkanolamides, such as stearamide diethanolamide distearate and stearamide monoethanolamide stearate;

vi)single-chain fatty alcohols comprising at least 20 carbon atoms, such as behenyl alcohol;

vii)long-chain (e.g., C₁₀-C₃₀) amine oxides, such as (C₁₀-C₃₀)alkyl-dimethylamine oxides, for example, stearyldimethylamine oxide;

viii)N,N-dihydrocarbyl (for example C₁₀-C₃₀, further, for example, C₁₂-C₂₂) amidobenzoic acids and their salts, such as N,N-di(C₁₆-C₁₈)amidobenzoic acid sold by the company Stefan Company;

ix)alcohols comprising from 27 to 48 carbon atoms and comprising one or two groups chosen from ether, thioether and sulphoxide groups, corresponding to formula (II):

Rₐ—X—[C₂H₃(OH)]—CH₂—Y—R_{b} (II)

in which Rₐ and R_{b}, which may be identical or different, are chosen from linear C₁₂ to C₂₄ groups;

X is chosen from an oxygen atom, a sulphur atom and sulphoxide and methylene groups;

Y is chosen from an oxygen atom, a sulphur atom and sulphoxide and methylene group;

the sum of the number of carbon atoms present in the groups Rₐ and R_{b} has a value ranging from 24 to 44 and, for example, from 28 to 40; when X or Y is sulphoxide, Y or X does not denote sulphur.

Examples of the compounds of formula (II) that can be used in accordance with the invention include those for which X is oxygen, Y is methylene and Rₐ and R_{b}, which may be identical or different, are chosen from radicals comprising 12 to 22 carbon atoms, it being possible for these compounds to be prepared according to the patent EP 457 688; and

x)coated and uncoated titanium oxides, micas and titanium micas.

The at least one agent chosen from nacreous and opacifying agents is, for example, chosen from families i), iv), vi) and ix) and is further, for example, chosen from ethylene glycol monostearate and distearate, distearyl ether, behenyl alcohol and 1-(hexadecyloxy)-2-octadecanol. The at least one agent chosen from nacreous and opacifying agents is further, for example, chosen from distearyl ether, behenyl alcohol and 1-(hexadecyloxy)-2-octadecanol.

According to the invention, the at least one agent chosen from nacreous and opacifying agents can be, for example, in an amount ranging from 0.1% to 15% by weight, such as from 0.5% to 10% by weight and further such as from 1% to 5% by weight relative to the total weight of the final composition.

The volume-average size of the silicone particles before introduction into the composition and/or in the final composition is generally, for example, ranging from 2 to 50 microns and further, for example, from 4 to 30 microns. This particle size is measured either with an optical microscope optionally followed by image processing, or by laser diffraction.

The silicones that may be used in accordance with the invention are, for example, insoluble in the composition and may be in the form of oils, waxes or gums.

According to the invention, the expression “water-insoluble silicone” means silicones that are insoluble in water at a concentration of greater than or equal to 0.1% by weight in water at 25° C., i.e., they do not form a transparent, macroscopically isotropic solution.

The silicones are defined in greater detail in Walter Noll's publication “Chemistry and Technology of Silicones” (1968) Academic Press.

According to the invention, all the silicones may also be used in unmodified form or in the form of solutions, emulsions, nanoemulsions or microemulsions.

The silicones of the invention are, for example, chosen from polydi(C1-C4)alkylsiloxanes, among which mention may be made, for example, of polydimethylsiloxanes comprising trimethylsilyl end groups.

Among these silicones comprising trimethylsilyl end groups that may be mentioned, in a non-limiting manner, are:

the oils of the Mirasil series sold by the company Rhodia Chimie, such as the oil Mirasil DM 500 000;

the oils of the 200 series from the company Dow Corning, such as DC200 Fluid 60 000 cSt, with a viscosity of 60 000 cSt; and

the Viscasil oils from General Electric and certain oils of the SF series (SF 96 and SF 18) from General Electric.

The polydimethylsiloxanes in accordance with the invention are, for example, oils with a viscosity ranging from 0.2 to 2.5 m²/s at 25° C., such as the oils of the DC200 series from Dow Corning, of the Silbione 70047 and 47 series and, for example, the oil Silbione 70 047 V 500 000 sold by the company Rhodia Chimie, or the silicone oil AK 300 000 from the company Wacker.

The viscosity of the silicones is measured, for example, at 25° C. according to ASTM standard 445 Appendix C.

The at least one silicone is, for example, used in an amount ranging from 0.01% to 20% by weight relative to the total weight of the composition. For example, this amount is from 0.05% to 15% by weight relative to the total weight of the composition and further, for example, 0.1% and 10% by weight relative to the total weight of the composition.

The cationic polymers that may be used according to the invention have a cationic charge density of less than 1 meq/g and, for example, from 0.1 to 1 meq/g and further, for example, from 0.2 to 0.9 meq/g. The charge density may be determined according to the Kjeldahl method. It is generally measured at a pH of the order of 3 to 9.

The cationic polymers that may be used in accordance with the present invention may be chosen from any of those already known as improving the cosmetic properties of the hair, such as those described in patent application EP-A-0 337 354 and in French patent applications FR-A-2 270 846, 2 383 660, 2 598 611, 2 470 596 and 2 519 863 and having a cationic charge density as defined above.

As used herein, “cationic polymer” refers to polymers chosen from polymers comprising at least one cationic group and polymers comprising at least one group which can be ionized to form cationic groups.

The cationic polymersmay, for example, be chosen from those comprising units comprising primary, secondary, tertiary and/or quaternary amine groups that may either form part of the main polymer chain, or may be borne by a side substituent that is directly attached to the main chain.

The cationic polymers used generally have a number-average or weight-average molar mass ranging from 500 to 5×10⁶ and, for example, from 10³ to 3×10⁶.

Among the cationic polymers that may be mentioned, for example, are polymers of polyamine, polymers of polyamino amide and polymers of polyquaternary ammonium. These polymers are known in the art.

The polymers of polyamine, polymers of polyamino amide and polymers of polyquaternary ammonium that may be used in accordance with the present invention, and that may, for example, be mentioned, are those described in French Patent Nos. 2 505 348 and 2 542 997. Among these polymers, mention may be made of:

(1)homopolymers or copolymers derived from acrylic or methacrylic esters or amides and comprising at least one of the units of the following formulae:

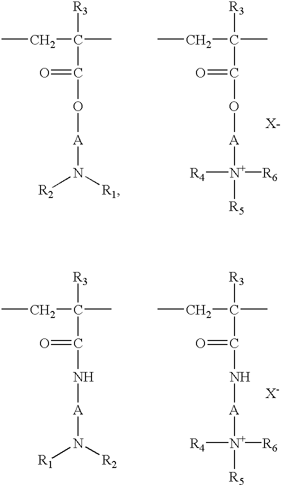

in which:

R₃, which may be identical or different, is chosen from a hydrogen atom and a CH₃ radical;

A, which may be identical or different, is chosen from linear and branched alkyl groups of 1 to 6 carbon atoms, such as 2 or 3 carbon atoms, and hydroxyalkyl groups of 1 to 4 carbon atoms;

R₄, R₅ and R₆, which may be identical or different, are chosen from alkyl groups comprising from 1 to 18 carbon atoms and benzyl radicals, for example, alkyl groups comprising from 1 to 6 carbon atoms;

R₁ and R₂, which may be identical or different, are chosen from a hydrogen atom and alkyl groups comprising from 1 to 6 carbon atoms, and, for example, methyl and ethyl groups;

X⁻ is an anion derived from an inorganic or organic acid, such as a methosulphate anion or an anion chosen from halides such as chloride and bromide.

Copolymers of family (1) can also comprise at least one unit derived from comonomers, which may be chosen from the family of acrylamides, methacrylamides, diacetone acrylamides, acrylamides and methacrylamides substituted on the nitrogen with at least one group chosen from lower (C₁-C₄) alkyls, acrylic acids, methacrylic acids, acrylic esters, methacrylic esters, vinyllactams, such as vinylpyrrolidone and vinylcaprolactam, and vinyl esters.

Thus, among these copolymers of family (1), mention may be made of:

the copolymers of acrylamide and of dimethylaminoethyl methacrylate quaternized with dimethyl sulphate or with a dimethyl halide,

the copolymers of acrylamide and of methacryloyloxyethyltrimethylammonium chloride described, for example, in patent application EP-A-080 976,

the copolymer of acrylamide and of methacryloyloxyethyltrimethylammonium methosulphate,

quaternized or non-quaternized vinylpyrrolidone/dialkylaminoalkyl acrylate or methacrylate copolymers, such as the products sold under the name “Gafquat” by the company ISP, such as “Gafquat 734” or “Gafquat 755”. These polymers are described in detail in French Patent Nos. 2 077 143 and 2 393 573,

dimethylaminoethyl methacrylate/vinylcaprolactam/vinylpyrrolidone terpolymers , such as the product sold under the name Gaffix VC 713 by the company ISP,

vinylpyrrolidone/methacrylamidopropyl copolymers, and

quaternized vinylpyrrolidone/dimethylaminopropylmethacrylamide copolymers, such as the product sold under the name “Gafquat HS 100” by the company ISP.

(2)cationic polysaccharides, such as celluloses and cationic galactomannan gums. Among the cationic polysaccharides that may be mentioned, for example, are cellulose ether derivatives comprising quaternary ammonium groups, cationic cellulose copolymers or cellulose derivatives grafted with a water-soluble quaternary ammonium monomer, and cationic galactomannan gums.

The cationic galactomannan gums are described, for example, in U.S. Pat. Nos. 3,589,578 and 4,031,307, such as guar gums comprising trialkylammonium cationic groups. For example, guar gums modified with a salt (e.g. chloride) of 2,3-epoxypropyltrimethylammonium may be used.

Such products are sold, for example, under the trade names Jaguar C13S, Jaguar C15, Jaguar C17 and Jaguar C162 by the company Meyhall.

The cellulose ether derivatives comprising quaternary ammonium groups, which are described in French Patent No.1 492 597. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described, for example, in U.S. Pat. No. 4,131,576, such as hydroxyalkylcelluloses, for instance hydroxymethylcelluloses, hydroxyethylcelluloses and hydroxypropylcelluloses grafted, for example, with a salt chosen from methacryloylethyltrimethylammonium salts, methacrylamidopropyltrimethylammonium salts and dimethyldiallylammonium salts.

(3)polymers comprising piperazinyl units and divalent alkylene or hydroxyalkylene radicals comprising straight or branched chains, optionally interrupted by at least one atom chosen from oxygen, sulphur and nitrogen atoms or by at least one aromatic or heterocyclic ring, as well as at least one of the oxidation and/or quaternization products of these polymers. Such polymers are described, for example, in French Patent Nos. 2 162 025 and 2 280 361;

(4)water-soluble polyamino amides prepared, for example, by polycondensation of an acidic compound with a polyamine; these polyamino amides being crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or with an oligomer resulting from the reaction of a difunctional compound, which is reactive with a bis-halohydrin, a bis-azetidinium, a bis-haloacyldiamine, a bis-alkyl halide, an epihalohydrin, a diepoxide or a bis-unsaturated derivative. The crosslinking agent can be used in proportions ranging from 0.025 to 0.35 mol per amine group of the polyamino amide. These polyamino amides can be alkylated or, if they comprise at least one tertiary amine function, they can be quaternized. Such polymers are described, for example, in French Patent Nos. 2 252 840 and 2 368 508;

(5)polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylenetriamine polymers in which the alkyl radical comprises from 1 to 4 carbon atoms and, for example, methyl, ethyl or propyl. Such polymers are described, for example, in French Patent No.1 583 363.

Among these derivatives, mention may be made, for example, of the adipic acid/dimethylaminohydroxypropyl/diethylenetriamine polymers.

(6)polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 8 carbon atoms. The molar ratio between the polyalkylene polyamine and the dicarboxylic acid may range from 0.8:1 to 1.4:1; the polyamino amide resulting therefrom may be reacted with epichlorohydrin in a molar ratio of epichlorohydrin relative to the secondary amine group of the polyamino amide ranging from 0.5:1 to 1.8:1. Such polymers are described, for example, in U.S. Pat. Nos. 3,227,615 and 2,961,347.

Other non-limiting examples of such derivatives include the adipic acid/epoxypropyl/diethylenetriamine copolymers sold, for example, under the name “Hercosett 57” by the company Hercules Inc. or under the name of “PD 170” or Delsette 101” by the company Hercules.

(7)cyclopolymers of alkyldiallylamine or of dialkyldiallylammonium, such as copolymers comprising, as main constituent of the chain, at least one unit corresponding to formula (III) or (IV):

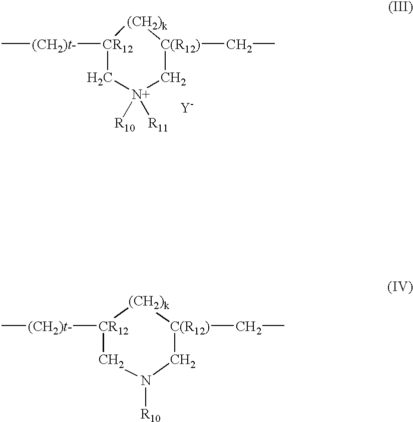

in which k and t are equal to 0 or 1, the sum k+t being equal to 1;

R₁₂ is chosen from a hydrogen atom and a methyl radical;

R₁₀ and R₁₁, which may be identical or different, are chosen from alkyl groups comprising from 1 to 6 carbon atoms, hydroxyalkyl groups in which the alkyl group, for example, comprises from 1 to 5 carbon atoms, lower C₁-C₄ amidoalkyl groups, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulphate, bisulphite, sulphate and phosphate. These polymers are described, for example, in French Patent No. 2 080 759 and in its Certificate of Addition 2 190 406.

In one embodiment, R10 and R11, which may be identical or different, for example, are chosen from alkyl groups comprising from 1 to 4 carbon atoms.

(8)The quaternary diammonium polymers comprising repeating units corresponding to the formula (V):

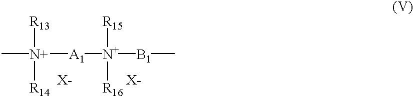

in which:

R₁₃, R₁₄, R₁₅ and R₁₆, which may be identical or different, are chosen from aliphatic, alicyclic and arylaliphatic radicals comprising from 1 to 20 carbon atoms and from lower hydroxyalkylaliphatic radicals, or R₁₃, R₁₄, R₁₅ and R₁₆, together or separately, constitute, with the nitrogen atoms to which they are attached, heterocycles optionally comprising a second hetero atom other than nitrogen, or R₁₃, R₁₄, R₁₅ and R₁₆ are chosen from linear and branched C₁-C₆ alkyl radicals substituted with at least one group chosen from nitrile, ester, acyl and amide groups and groups of —CO—O—R₁₇—D and —CO—NH—R₁₇—D, wherein R₁₇ is chosen from alkylene groups and D is chosen from quaternary ammonium groups;

A₁ and B₁, which may be identical or different, are chosen from linear and branched, saturated and unsaturated polymethylene groups comprising from 2 to 20 carbon atoms. The polymethylene groups may comprise, linked to or intercalated in the main chain, at least one entity chosen from aromatic rings, oxygen and sulphur atom and sulphoxide, sulphone, disulphide, amino, alkylamino, hydroxyl, quaternary ammonium, ureido, amide and ester groups; and

X⁻ is an anion chosen from anions derived from inorganic acids and organic acids.

A₁, R₁₃ and R₁₅ may optionally form, with the two nitrogen atoms to which they are attached, a piperazine ring. In addition, if A₁ is a radical chosen from linear and branched, saturated and unsaturated alkylene and hydroxyalkylene radicals, B₁ can also denote a group (CH₂)ₙ—CO—D—OC—(CH₂)ₙ—, wherein n ranges from 1 to 100, such as from 1 to 50,

D is chosen from:

a)a glycol residue of formula: —O—Z—O—, wherein Z is chosen from linear and branched hydrocarbon-based radicals and a group corresponding to one of the following formulae:

—(CH₂—CH₂—O)ₓ—CH₂—CH₂—

—[CH₂—CH(CH₃)—O]_{y}—CH₂—CH(CH₃)—

wherein x and y, which may be identical or different, are each an integer ranging from 1 to 4, representing a defined and unique degree of polymerization or any number ranging from 1 to 4 representing an average degree of polymerization;

b)a bis-secondary diamine residue such as a piperazine derivative;

c)a bis-primary diamine residue of formula: —NH—Y—NH—, wherein Y is chosen from linear and branched hydrocarbon radicals, and the divalent radical —CH₂—CH₂—S—S—CH₂—CH₂—; and

d)a ureylene group of formula: —NH—CO—NH—.

In one embodiment, X⁻ is an anion such as chloride or bromide.

These polymers may have a number-average molecular mass ranging from 1000 to 100,000.

These polymers are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Further, according to the present invention, polymers can comprise repeating units corresponding to the formula (a):

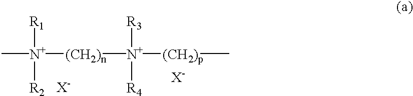

in which R1, R2, R3 and R4, which may be identical or different, are chosen from alkyl and hydroxyalkyl radicals comprising from 1 to 4 carbon atoms, n and p, which may be identical or different, are integers ranging from 2 to 20, and X⁻ is an anion chosen from anions derived from inorganic acids and organic acids.

(9)polyquaternary ammonium polymers comprising repeating units of formula (VI):

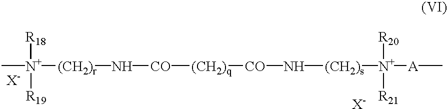

in which:

R₁₈, R₁₉, R₂₀ and R₂₁, which may be identical or different, are chosen from a hydrogen atom and methyl, ethyl, propyl, β-hydroxyethyl, β-hydroxypropyl and —CH₂CH₂(OCH₂CH₂)ₚOH radicals,

wherein p is equal to 0 or to an integer ranging from 1 to 6, with the proviso that R₁₈, R₁₉, R₂₀ and R₂₁ do not simultaneously represent a hydrogen atom,

r and s, which may be identical or different, are chosen from integers ranging from 1 to 6,

q is equal to 0 or to an integer from 1 to 34,

X⁻ is an anion such as a halide,

A is chosen from divalent radicals such as —CH₂—CH₂—O—CH₂—CH₂—.

Such polymers are described, for example, in patent application EP-A-122 324.

(10) quaternary polymers of vinylpyrrolidone and of vinylimidazole, such as the products sold under the name Luviquat® HOL by the company BASF.(11) polyamines, for instance Polyquart® H sold by Cognis under the reference name “Polyethylene glycol (15) tallow polyamine” in the CTFA dictionary.

(12)Crosslinked methacryloyloxy(C₁-C₄)alkyltri(C₁-C₄)alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, such as methylenebisacrylamide.

Other cationic polymers, which can be used in the context of the invention, are chosen from cationic proteins and cationic protein hydrolysates, polyalkyleneimines, such as polyethyleneimines, polymers comprising units chosen from vinylpyridine units and vinylpyridinium units, condensates of polyamines and of epichlorohydrin, quaternary polyureylenes and chitin derivatives.

Among all the cationic polymers that may be used in the context of the present invention, non-limiting examples include quaternary cellulose ether derivatives, such as the products sold under the name “JR 400” by the company Amerchol, quaternary polymers of vinylpyrrolidone and of vinylimidazole, crosslinked copolymers of methacryloyloxy(C₁-C₄)alkyltri(C₁-C₄)alkylammonium salts, and mixtures thereof.

The amphoteric polymers, which may be used in accordance with the present invention, may be chosen from polymers comprising units K and M randomly distributed in the polymer chain, in which K is a unit derived from a monomer comprising at least one basic nitrogen atom and M is a unit derived from an acidic monomer comprising at least one carboxylic or sulphonic group, or K and M may be chosen from groups derived from zwitterionic carboxybetaine or sulphobetaine monomers.

K and M may also be chosen from a cationic polymer chain comprising at least one group chosen from primary, secondary, tertiary and quaternary amine groups, in which at least one of the amine groups bears a carboxylic or sulphonic group linked via a hydrocarbon-based radical, or K and M form part of a chain of a polymer comprising an α,β-dicarboxylic ethylene unit in which one of the carboxylic groups has been made to react with a polyamine comprising at least one amine chosen from primary and secondary amine groups.

The amphoteric polymers corresponding to the above definition, for example, are chosen from the following polymers:

(1)polymers resulting from the copolymerization of at least one monomer derived from a vinyl compound bearing a carboxylic group such as acrylic acid, methacrylic acid, maleic acid, α-chloroacrylic acid, and at least one basic monomer derived from a substituted vinyl compound comprising at least one basic atom, such as dialkylaminoalkyl methacrylate and acrylate, dialkylaminoalkylmethacrylamide and -acrylamide. Such compounds are described, for example, in U.S. Pat. No. 3,836,537.

Mention may also be made of the sodium acrylate/acrylamidopropyltrimethylammonium chloride copolymer sold under the name Polyquart KE 3033 by the company Cognis.

The vinyl compound may also be a dialkyldiallylammonium salt such as dimethyldiallylammonium salt (for example chloride). The copolymers of acrylic acid and of the latter monomer are sold under the name Merquat Plus 3330 by the company Nalco.

(2) polymers comprising units derived from:

a) at least one monomer chosen from acrylamides and methacrylamides substituted on the nitrogen with an alkyl radical,

b) at least one acidic comonomer comprising at least one reactive carboxylic group, and

c) at least one basic comonomer such as esters comprising substituents chosen from primary, secondary, tertiary and quaternary amine substituents of acrylic and methacrylic acids and the product of quaternization of dimethylaminoethyl methacrylate with dimethyl or diethyl sulphate.

In one embodiment, the N-substituted acrylamides or methacrylamides according to the invention are, for example, groups in which the alkyl radicals comprise from 2 to 12 carbon atoms, such as N-ethylacrylamide, N-tert-butylacrylamide, N-tertoctylacrylamide, N-octylacrylamide, N-decylacrylamide, N-dodecylacrylamide and the corresponding methacrylamides.

The acidic comonomers are chosen, for example, from acrylic acids, methacrylic acids, crotonic acids, itaconic acids, maleic acids and fumaric acids and alkyl monoesters, comprising 1 to 4 carbon atoms, of maleic or fumaric acids or anhydrides.

The basic comonomers are chosen, for example, from aminoethyl, butylaminoethyl, N,N′-dimethylaminoethyl and N-tert-butylaminoethyl methacrylates. The copolymers having the CTFA (4th edition, 1991) name octylacrylamide/acrylates/butylaminoethyl methacrylate copolymer can, for example, also be used.

(3) crosslinked and alkylated polyamino amides partially or totally derived from polyamino amides of general formula:

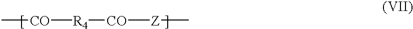

in which R₄ is chosen from a divalent radical derived from saturated dicarboxylic acid, mono- and dicarboxylic aliphatic acids comprising an ethylenic double bond, an ester of a lower alkanol, comprising from 1 to 6 carbon atoms, of these acids and a radical derived from the addition of any one of the said acids to amines chosen from bis(primary) and bis(secondary) amines, and Z is chosen from bis(primary), mono- and bis(secondary) polyalkylene-polyamine radicals and, for example, Z represents:

a) in proportions of from 60 to 100 mol %, the radical

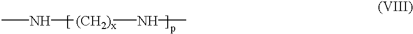

wherein x=2 and p=2 or 3, or x=3 and p=2, this radical being derived from a compound chosen from diethylenetriamine, triethylenetetraamine and dipropylenetriamine;

b) in proportions of from 0 to 40 mol %, the radical (VIII) above in which x=2 and p=1 and which is derived from a compound chosen from ethylenediamine and piperazine:

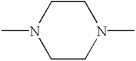

c) in proportions of from 0 to 20 mol %, the —NH—(CH₂)₆—NH— radical, which is derived from hexamethylenediamine, these polyamino amides can be crosslinked by addition of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

In one embodiment, the saturated carboxylic acids are, for example, chosen from acids comprising from 6 to 10 carbon atoms, such as adipic acid, 2,2,4-trimethyladipic acid and 2,4,4-trimethyladipic acid, terephthalic acid and acids comprising an ethylenic double bond such as acrylic acid, methacrylic acid and itaconic acid.

The alkane sultones used in the alkylation are chosen, for example, from propane sultones and butane sultones, and the salts of the alkylating agents can, for example, be chosen from sodium and potassium salts.

(4)polymers comprising zwitterionic units of formula:

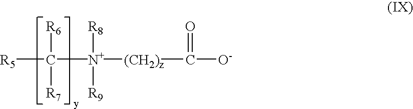

in which R₅ is chosen from polymerizable unsaturated groups, such as acrylate, methacrylate, acrylamide and methacrylamide groups,

y and z, which may be identical or different, are chosen from integers ranging from 1 to 3,

R₆ and R₇, which may be identical or different, are chosen from a hydrogen atom, methyl, ethyl and propyl groups,

R₈ and R₉, which may be identical or different, are chosen from a hydrogen atom and alkyl radicals such that the sum of the carbon atoms in R₈ and R₉ does not exceed 10.

The polymers comprising such units can also comprise units derived from non-zwitterionic monomers such as monomers chosen from dimethyl and diethylaminoethyl acrylates and methacrylates, alkyl acrylates, methacrylates, acrylamides, methacrylamides and vinyl acetate.

By way of example, mention may be made of the copolymer of butyl methacrylate/dimethylcarboxymethylammonioethyl methacrylate.

(5)polymers derived from chitosan comprising monomer units corresponding to formulae (X), (XI) and (XII) below:

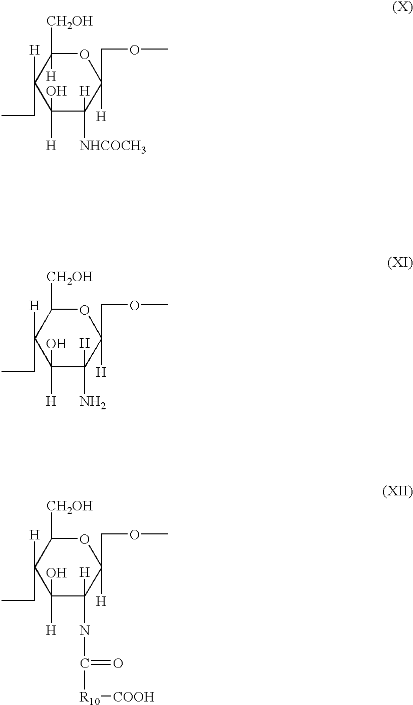

the unit (X) being present in proportions ranging from 0 to 30%, the unit (XI) in proportions ranging from 5% to 50% and the unit (XII) in proportions ranging from 30% to 90%, and wherein in the unit (XII), R₁₀ is a radical of formula:

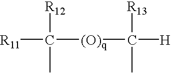

wherein q is equal to 0 or 1;

if q=0, R₁₁, R₁₂ and R₁₃, which may be identical or different, are chosen from a hydrogen atom, methyl, hydroxyl, acetoxy and amino residues, monoalkylamine residues and dialkylamine residues, which are optionally interrupted by at least one nitrogen atom and/or optionally substituted with at least one group chosen from amine, hydroxyl, carboxyl, alkylthio and sulphonic groups, and alkylthio residues in which the alkyl group bears an amino residue, at least one of the radicals R₁₁, R₁₂ and R₁₃ being, in this case, a hydrogen atom;

or, if q=1, R₁₁, R₁₂ and R₁₃, which may be identical or different, are chosen from a hydrogen atom, and the salts formed by these compounds with bases or acids.

(6)polymers derived from the N-carboxyalkylation of chitosan, such as N-carboxymethylchitosan and N-carboxybutylchitosan.

(7)polymers corresponding to the general formula (XIII) as described, for example, in French Patent No. 1 400 366:

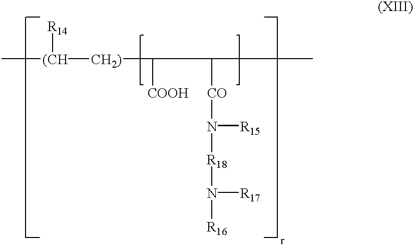

in which R₁₄ is chosen from a hydrogen atom, and CH₃O, CH₃CH₂O and phenyl radicals,

R₁₅ is chosen from hydrogen and lower alkyl radicals such as methyl and ethyl,

R₁₆ is chosen from hydrogen and lower alkyl radicals such as methyl and ethyl,

R₁₇ is chosen from lower alkyl radicals such as methyl and ethyl radicals corresponding to the formula: —R₁₈—N(R₁₆)₂, wherein R₁₈ is chosen from —CH₂—CH₂—, —CH₂—CH₂—CH₂— and —CH₂—CH(CH₃)— groups, and R₁₆ is chosen from a hydrogen atom and lower alkyl radicals such as methyl and ethyl, and the higher homologues of these radicals comprising up to 6 carbon atoms,

r is chosen such that the number-average molecular weight of said polymer ranges from 500 to 6,000,000, such as from 1,000 to 1,000,000.

(8)amphoteric polymers of the type —D—X—D—X— chosen from:

a)polymers obtained by the action of chloroacetic acid or sodium chloroacetate on compounds comprising at least one unit of formula:

—D—X—D—X—D— (XIV)

wherein D is a radical

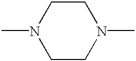

and X is chosen from the symbols E and E′, wherein E and E′, which may be identical or different, are chosen from divalent alkylene radicals comprising at least one chain chosen from straight and branched chains comprising up to 7 carbon atoms in the main chain, wherein said divalent alkylene radicals are optionally substituted with at least one hydroxyl group. E or E′ can additionally comprise at least one atom chosen from oxygen, nitrogen and sulphur atoms, and 1 to 3 rings chosen from aromatic and heterocyclic rings. The oxygen, nitrogen and sulphur atoms can be present in the form of at least one group chosen from ether, thioether, sulphoxide, sulphone, sulphonium, alkylamine, alkenylamine, hydroxyl, benzylamine, amine oxide, quaternary ammonium, amide, imide, alcohol, ester and urethane groups;

b)polymers of formula:

—D—X—D—X— (XV)

wherein D is a radical

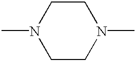

and X is chosen from the symbols E and E′ and wherein at least one X is chosen from E′; E having the meaning given above and E′ being chosen from divalent alkylene radicals comprising at least one chain chosen from straight and branched chains comprising up to 7 carbon atoms in the main chain, wherein said divalent alkylene radicals are optionally substituted with at least one hydroxyl radical. E′ can also comprise at least one nitrogen atom substituted with an alkyl chain, which is optionally interrupted by an oxygen atom, wherein said alkyl chain comprises at least one functional group chosen from carboxyl functional groups and hydroxyl functional groups, and wherein the alkyl chain is betainized by reaction with a reactant chosen from chloroacetic acid and sodium chloroacetate.

(9)(C₁-C₅)alkyl vinyl ether/maleic anhydride copolymers partially modified by semiamidation with an N,N-dialkylaminoalkylamine such as N,N-dimethylaminopropylamine or by semiesterification with an N,N-dialkanolamine. These copolymers can also comprise other vinyl comonomers such as vinylcaprolactam.

The amphoteric polymers according to the invention are, in some embodiments, those of family (1).

According to the invention, the at least one polymer chosen from cationic and amphoteric polymers may be in an amount ranging, for example, from 0.001% to 20% by weight, further such as from 0.01% to 10% by weight and even further such as from 0.05% to 1% by weight relative to the total weight of the final composition.

According to one embodiment of the present invention, the compositions further comprise at least one additional silicone other than polydialkylsiloxanes comprising trimethylsilyl end groups or another agent that is beneficial for keratin materials, such as the hair, for example, esters of C₁-C₃₀ carboxylic acids and of C₁-C₃₀ mono- or polyhydroxylated alcohols other than the nacreous agents mentioned above, plant, animal, mineral or synthetic oils, waxes, ceramides and pseudoceramides.

According to the invention, all the silicones may also be used in unmodified form or in the form of solutions, emulsions, nanoemulsions or microemulsions.

Examples of the additional silicones in accordance with the invention include, for example:

non-volatile silicones chosen from the family of polyalkylsiloxanes comprising dimethylsilanol end groups, such as oils with a viscosity ranging from 0.2 to 2.5 m²/s at 25° C., and aminosilicones such as amodimethicones and trimethylsilylamodimethicones.

According to the invention, the additional silicones or the other additional beneficial agents can be in an amount ranging, for example, from 0.001% to 20% by weight, further, for example, from 0.01% to 10% by weight and even further, for example, from 0.1% to 5% by weight relative to the total weight of the composition.

The compositions of the invention can also comprise at least one surfactant, which is generally present in an amount ranging from 0.01% to 50% by weight, such as from 0.1% to 40% and further such as from 0.5% to 30% relative to the total weight of the composition.

The at least one surfactant may be chosen from anionic, amphoteric, nonionic and cationic surfactants.

The at least one surfactant that is suitable for carrying out the present invention is, for example, chosen from:

(i)Anionic surfactants:

As examples of anionic surfactants, which can be used, alone or as mixtures, in the context of the present invention, mention may be made, for example, of salts (such as alkaline salts, for example, sodium salts, ammonium salts, amine salts, amino alcohol salts or magnesium salts) of the following compounds: alkyl sulphates, alkyl ether sulphates, alkylamidoether sulphates, alkylarylpolyether sulphates, monoglyceride sulphates; alkyl sulphonates, alkyl phosphates, alkylamide sulphonates, alkylaryl sulphonates, α-olefin sulphonates, paraffin sulphonates; alkyl sulphosuccinates, alkyl ether sulphosuccinates, alkylamide sulphosuccinates; alkyl sulphosuccinamates; alkyl sulphoacetates; alkyl ether phosphates; acyl sarcosinates; acyl isethionates and N-acyltaurates. The alkyl or acyl radical of all of these various compounds, for example, comprise from 8 to 24 carbon atoms, and the aryl radical is chosen, for example, from phenyl and benzyl groups. Among the anionic surfactants, which can also be used, mention may also be made of fatty acid salts such as the salts of oleic, ricinoleic, palmitic and stearic acids, coconut oil acid or hydrogenated coconut oil acid; acyl lactylates in which the acyl radical comprises from 8 to 20 carbon atoms. Weakly anionic surfactants can also be used, such as alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, for example, those comprising from 2 to 50 ethylene oxide groups, and mixtures thereof.

Among the anionic surfactants, for example, alkyl sulphate salts and alkyl ether sulphate salts and mixtures thereof can be used.

(ii) Nonionic surfactants:

The nonionic surfactants are compounds that are well known (see, for example, in this respect “Handbook of Surfactants” by M. R. Porter, published by Blackie & Son (Glasgow and London), 1991, pp. 116-178). They can be chosen, for example, from polyethoxylated, polypropoxylated and polyglycerolated fatty acids, alkylphenols, α-diols and alcohols comprising a fatty chain comprising, for example, from 8 to 18 carbon atoms, it being possible for the number of ethylene oxide or propylene oxide groups to range, for example, from 2 to 50 and for the number of glycerol groups to range, for example, from 2 to 30. Mention may also be made of copolymers of ethylene oxide and of propylene oxide, condensates of ethylene oxide and of propylene oxide with fatty alcohols; polyethoxylated fatty amides, for example, those comprising from 2 to 30 mol of ethylene oxide, polyglycerolated fatty amides comprising on average from 1 to 5, and such as from 1.5 to 4, glycerol groups; polyethoxylated fatty amines such as those comprising from 2 to 30 mol of ethylene oxide; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides. It may be noted that the alkylpolyglycosides constitute nonionic surfactants that can be used in the context of the present invention.

(iii) Amphoteric surfactants:

The amphoteric surfactants can be chosen, for example, from aliphatic secondary and tertiary amine derivatives in which the aliphatic radical is a chosen from linear and branched chains comprising from 8 to 22 carbon atoms and comprising at least one water-soluble anionic group (for example carboxylate, sulphonate, sulphate, phosphate or phosphonate); mention may also be made of (C₈-C₂₀)alkylbetaines, sulphobetaines, (C₈-C₂₀)alkylamido(C₁-C₆)alkylbetaines or (C₈-C₂₀)alkylamido(C₁-C₆)alkylsulphobetaines.

Among the (C₈-C₂₀)alkylamido(C₁-C₆)alkylbetaines that may be mentioned is the cocoamidopropylbetaine sold, for example, by Goldschmidt under the name Tegobetaine F50.

Among the amine derivatives, mention may be made of the products sold under the name Miranol, as described, for example, in U.S. Pat. Nos. 2,528,378 and 2,781,354 and having the structures of:

R₂—CONHCH₂CH₂—N⁺(R₃)(R₄)(CH₂COO⁻) (2)

in which: R₂ is chosen from alkyl radicals derived from an acid R₂—COOH present in hydrolyzed coconut oil, and heptyl, nonyl and undecyl radicals, R₃ is a β-hydroxyethyl group and R₄ is a carboxymethyl group;

and of

R₅—CONHCH₂CH₂—N(B)(C) (3)

wherein B represents —CH₂CH₂OX′, C represents —(CH₂)_{z}—Y′, with z=1 or 2, X′ is chosen from the —CH₂CH₂—COOH group and a hydrogen atom, Y′ is chosen from the —COOH and the —CH₂—CHOH—SO₃H radicals, R₅ is chosen from alkyl radicals of an acid R₅—COOH present in coconut oil or in hydrolyzed linseed oil, alkyl radicals, such as C₇, C₉, C₁₁ and C₁₃ alkyl radicals, a C₁₇ alkyl radical and its iso form, and an unsaturated C₁₇ radical.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, and cocoamphodipropionic acid.

By way of example, mention may be made of the cocoamphodiacetate sold under the trade name Miranol C2M concentrated by the company Rhodia Chimie.

(iv)The cationic surfactants may be chosen from:

A)the quaternary ammonium salts of general formula (XVI) below:

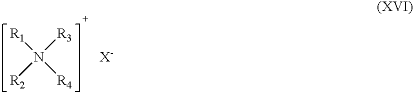

in which X⁻ is an anion chosen from halides (chloride, bromide and iodide), (C₂-C₆)alkyl sulphates, such as methyl sulphate, phosphates, alkyl and alkylaryl sulphonates, and anions derived from organic acids, such as acetate and lactate, and

a)the radicals R₁ to R₃, which may be identical or different, are chosen from linear and branched aliphatic radical comprising from 1 to 4 carbon atoms, and aromatic radicals such as aryl and alkylaryl. The aliphatic radicals can comprise at least one hetero atom such as oxygen, nitrogen, sulphur and halogen. The aliphatic radicals are chosen, for example, from alkyl, alkoxy and alkylamide radicals,

R₄ is chosen from linear and branched alkyl radicals comprising from 16 to 30 carbon atoms.

The cationic surfactant is, for example, a behenyltrimethylammonium salt (for example chloride).

b)the radicals R₁ and R₂, which may be identical or different, are chosen from linear and branched aliphatic radicals comprising from 1 to 4 carbon atoms, and aromatic radicals such as aryl and alkylaryl. The aliphatic radicals can comprise at least one hetero atom such as oxygen, nitrogen, sulphur and halogen. The aliphatic radicals are chosen, for example, from alkyl, alkoxy, alkylamide and hydroxyalkyl radicals comprising from about 1 to 4 carbon atoms;

R₃ and R₄, which may be identical or different, are chosen from linear and branched alkyl radicals comprising from 12 to 30 carbon atoms, the alkyl radical may comprise at least one ester or amide function.

R₃ and R₄ are chosen, for example, from (C₁₂-C₂₂)alkylamido(C₂-C₆)alkyl and (C₁₂-C₂₂)alkylacetate radicals.

The cationic surfactant is, for example, a stearamidopropyldimethyl(myristyl acetate)ammonium salt (for example chloride);

B)—the quaternary ammonium salts of imidazolinium, such as that of formula (XVII) below:

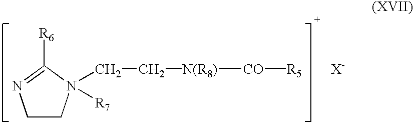

in which R₅ is chosen from alkenyl and alkyl radicals comprising from 8 to 30 carbon atoms, for example fatty acid derivatives of tallow,

R₆ is chosen from a hydrogen atom, C₁-C₄ alkyl radicals and alkenyl and alkyl radicals comprising from 8 to 30 carbon atoms,

R₇ is chosen from C₁-C₄ alkyl radicals,

R₈ is chosen from a hydrogen atom and C₁-C₄ alkyl radicals, and

X⁻ is an anion chosen from halides, phosphates, acetates, lactates, alkyl sulphates, alkyl sulphonates and alkylaryl sulphonates.

In one embodiment, R₅ and R₆ are, for example, a mixture of radicals chosen from alkenyl and alkyl radicals comprising from 12 to 21 carbon atoms, such as fatty acid derivatives of tallow, R₇ is methyl and R₈ is hydrogen. Such a product is, for example, Quaternium-27 (CTFA 1997) or Quaternium-83 (CTFA 1997), which are sold under the names “Rewoquat” W75, W90, W75PG and W75HPG by the company Witco.

C)—the diquaternary ammonium salts of formula (XVIII):

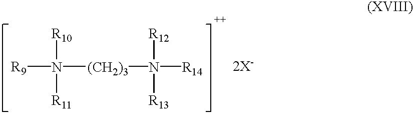

in which R₉ is chosen from aliphatic radicals comprising from 16 to 30 carbon atoms,

R₁₀, R₁₁, R₁₂, R₁₃ and R₁₄, which may be identical or different, are chosen from hydrogen and alkyl radicals comprising from 1 to 4 carbon atoms, and

X⁻ is an anion chosen from halides, acetates, phosphates, nitrates and methyl sulphates. Such diquaternary ammonium salts include, for example, propanetallowdiammmonium dichloride; and

D)—the quaternary ammonium salts comprising at least one ester function, of formula (XIX) below:

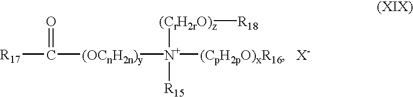

in which:

R₁₅ is chosen from C₁-C₆ alkyl radicals and C₁-C₆ hydroxyalkyl and dihydroxyalkyl radicals;

R₁₆ is chosen from:

a radical

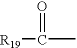

linear and branched, saturated and unsaturated C₁-C₂₂ hydrocarbon-based radicals R₂₀, and

a hydrogen atom,

R₁₈ is chosen from:

a radical

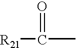

linear and branched, saturated and unsaturated C₁-C₆ hydrocarbon-based radicals R₂₂, and

a hydrogen atom,

R₁₇, R₁₉ and R₂₁, which may be identical or different, are chosen from linear and branched, saturated and unsaturated C₇-C₂₁ hydrocarbon-based radicals;

n, p and r, which may be identical or different, are chosen from integers ranging from 2 to 6;

y is chosen from integers ranging from 1 to 10;

x and z, which may be identical or different, are chosen from integers ranging from 0 to 10;

X⁻ is an anion chosen from simple and complex, organic and inorganic anions;

with the proviso that the sum x+y+z is from 1 to 15, that when x is 0, then R₁₆ is R₂₀ and that when z is 0, then R₁₈ is R₂₂.

In one embodiment, the ammonium salts of formula (XX) can be used, in which:

R₁₅ is chosen from methyl and ethyl radicals,

x and y are equal to 1;

z is equal to 0 or 1;

n, p and r are equal to 2;

R₁₆ is chosen from:

a radical

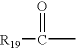

methyl, ethyl and C₁₄-C₂₂ hydrocarbon-based radicals, and

a hydrogen atom;

R₁₇, R₁₉ and R₂₁, which may be identical or different, are chosen from linear and branched, saturated and unsaturated C₇-C₂₁ hydrocarbon-based radicals;

R₁₈ is chosen from:

a radical

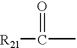

and

a hydrogen atom.

Such compounds are sold, for example, under the names Dehyquart by the company Cognis, Stepanquat by the company Stepan, Noxamium by the company Ceca, and Rewoquat WE 18 by the company Rewo-Witco.

Among the quaternary ammonium salts, examples are behenyltrimethylammonium chloride and stearamidopropylmethyl(myristyl acetate)ammonium chloride, sold under the name “Ceraphyl 70” by the company Van Dyk, and Quaternium-27 or Quaternium-83 sold by the company Witco.

In the compositions in accordance with the invention, mixtures of surfactants, such as mixtures of anionic surfactants, mixtures of anionic surfactants and of at least one surfactant chosen from amphoteric, cationic and nonionic surfactants, and mixtures of cationic surfactants with at least one surfactant chosen from nonionic and amphoteric surfactants may be used. One mixture, for example, is a mixture comprising at least one anionic surfactant and at least one amphoteric surfactant.

The composition of the invention may also comprise at least one additive chosen from thickeners, fragrances, preserving agents, silicone and non-silicone sunscreens, anionic and nonionic polymers, non-cationic proteins, non-cationic protein hydrolysates, 18-methyl eicosanoic acid, hydroxy acids, vitamins, provitamins such as panthenol, and any other additive conventionally used in cosmetics that does not affect the properties of the compositions according to the invention.

These additives are optionally present in the composition according to the invention in proportions that can range from 0.001% to 20% by weight relative to the total weight of the composition. The precise amount of each additive is readily determined by a person skilled in the art on the basis of its nature and its function.

The physiologically and cosmetically acceptable medium may consist solely of water or of a mixture of water and a cosmetically acceptable solvent such as a C₁-C₄ lower alcohol, for instance ethanol, isopropanol, tert-butanol or n-butanol; alkylene glycols, for instance propylene glycol, and glycol ethers.

For example, the composition comprises from 50% to 95% by weight of water relative to the total weight of the composition, and further, for example, from 65% to 90% by weight of water relative to the total weight of the composition.

The compositions according to the invention can have a final pH generally ranging from 3 to 10. For example, this pH ranges from 4 to 8. Adjusting the pH to the desired value may be performed conventionally by adding a base (organic or mineral base) to the composition, for example aqueous ammonia or a primary, secondary or tertiary (poly)amine, for instance monoethanolamine, diethanolamine, triethanolamine, isopropanolamine or 1,3-propanediamine, or by adding a mineral or organic acid, such as carboxylic acid such as, for example, citric acid.

The compositions in accordance with the invention may be used, for example, for washing or treating keratin materials such as the hair, the skin, the eyelashes, the eyebrows, the nails, the lips or the scalp.

The compositions according to the invention may be detergent compositions such as shampoos, shower gels and bubble baths. In this embodiment of the invention, the compositions comprise at least one washing base, which is generally aqueous.

The at least one washing base comprises at least one detergent surfactant. The at least one surfactant may be chosen, without discrimination, alone or as mixtures, from the anionic, amphoteric, nonionic and cationic surfactants as defined above.

In the compositions in accordance with the invention, at least one anionic surfactant or mixtures of at least one anionic surfactant and of at least one surfactant chosen from amphoteric surfactants and nonionic surfactants can, for example, be used.

In one embodiment, a mixture, for example, is a mixture comprising at least one anionic surfactant and at least one amphoteric surfactant.

For example, one can use an anionic surfactant chosen from sodium, triethanolamine or ammonium (C₁₂-C₁₄)alkyl sulphates, sodium, triethanolamine and ammonium (C₁₂-C₁₄)alkyl ether sulphates oxyethylenated with 2.2 mol of ethylene oxide, sodium cocoyl isethionate and sodium α-(C₁₄-C₁₆)olefin sulphonate, and mixtures thereof with:

either an amphoteric surfactant such as the amine derivatives known as disodium cocoamphodipropionate and sodium cocoamphopropionate sold, for example, by the company Rhodia Chimie under the trade name “Miranol C2M CONC” as an aqueous solution comprising 38% active material, or under the name Miranol C32; or

an amphoteric surfactant of zwitterionic type, such as alkylbetaines and alkylamidobetaines and, for example, the cocobetaine sold under the name “Dehyton AB 30” as an aqueous solution comprising 32% AM by the company Cognis, or the cocoamidopropylbetaine sold, for example, by Goldschmidt under the name Tegobetaine F50.

The quantity and quality of the washing base are those that are sufficient to be able to give the final composition satisfactory foaming power and/or detergent power.

These detergent compositions are, for example, foaming and the foaming power of the compositions according to the invention, characterized by a foam height, is generally greater than 75 mm, such as greater than 100 mm, measured according to the modified Ross-Miles method (NF T 73-404/IS696).

The modifications to the method are the following:

The measurement is performed at a temperature of 22° C. with osmosed water. The concentration of the solution is 2 g/l. The height of the drop is 1 m. The amount of composition that is dropped is 200 ml. These 200 ml of composition fall into a measuring cylinder 50 mm in diameter and containing 50 ml of the test composition. The measurement is taken 5 minutes after stopping the flow of the composition.

Thus, according to the invention, the washing base can be in an amount ranging from 3% to 50% by weight, such as from 6% to 35% by weight and further such as from 8% to 25% by weight relative to the total weight of the final composition.

Another aspect of the invention is a process for treating a keratin material such as the skin or the hair, characterized in that the process comprises applying to the keratin material a cosmetic composition as defined above, and then optionally rinsing it out with water.

Thus, this process according to the invention can allow the maintenance of the hairstyle and the treatment, care, washing or removal of makeup of the skin, the hair or any other keratin material.

The compositions of the invention may also be in the form of rinse-out or leave-in conditioners, permanent-waving, hair-straightening, dyeing or bleaching compositions, or in the form of rinse-out compositions to be applied before or after dyeing, bleaching, permanent-waving or straightening the hair or between the two steps of a permanent-waving or hair-straightening operation.

When the composition is in the form of a conditioner, such as a rinse-out conditioner, it, for example, comprises at least one cationic surfactant, and its concentration is generally ranging from 0.1% to 10% by weight, and such as from 0.5% to 5% by weight relative to the total weight of the composition.

The compositions of the invention may also be in the form of washing compositions for the skin, such as in the form of bath or shower solutions or gels or makeup-removing products.

The compositions according to the invention may also be in the form of aqueous or aqueous-alcoholic lotions for skincare and/or haircare.

The cosmetic compositions according to the invention may be in the form of a gel, a milk, a cream, an emulsion, a thickened lotion or a mousse and may be used for the skin, the nails, the eyelashes, the lips and, for example, the hair.

The compositions may be packaged in various forms, such as in vaporizers, pump-dispenser bottles or in aerosol containers to allow an application of the composition in vaporized form or in the form of a mousse. Such packaging forms are indicated, for example, when it is desired to obtain a spray, a lacquer or a mousse for treating keratin materials, such as the hair.

The compositions may be prepared by making a pre-emulsion of the silicone with at least one of the surfactants and water. This pre-emulsion is then added to the rest of the ingredients.

Throughout the text hereinabove and hereinbelow, the percentages expressed are on a weight basis.

The invention will now be illustrated more fully with the aid of the examples that follow, which cannot be considered as limiting it to the embodiments described.

In the examples, AM means active material.

### EXAMPLES 1 to 4

Shampoos in accordance with the invention, comprising the composition below, were prepared:

| Composition | Example 1 | Example 2 | Example 3 | Example 4 |
|---|---|---|---|---|
| Sodium lauryl ether | 16.0 g AM | 17.0 g AM | 16.0 g AM | 17.0 g AM |
| sulphate containing 2.2 | | | | |
| mol of ethylene oxide | | | | |
| Cocoylbetaine | 2.4 g AM | | 2.4 g AM | |
| Cocamidopropylbetaine | | | | 2.5 g AM |
| Sodium cocamidoethyl(N- | | 3.0 g AM | | |
| hydroxyethyl-N-carboxy- | | | | |
| methyl)glycinate (Miranol | | | | |
| C2M conc from Rhodia | | | | |
| Chimie) | | | | |
| Methacrylic acid/ethyl | 1.1 g AM | 1.0 g AM | 0.8 g AM | 1.5 g AM |
| acrylate crosslinked | | | | |
| copolymer as an aqueous | | | | |
| emulsion containing 30% | | | | |
| AM, sold under the name | | | | |
| Carbopol Aqua SF1 by the | | | | |
| company Noveon | | | | |
| Polydimethylsiloxane of | 2.5 g | 2.2 g | | |
| viscosity 60 000 cSt, sold | | | | |
| under the name DC200 | | | | |
| Fluid 60 000 cSt by the | | | | |
| company Dow Corning | | | | |
| Polydimethylsiloxane of | | | 2.5 g | 1.8 g |
| viscosity 300 000 cSt, sold | | | | |
| under the name DC200 | | | | |
| Fluid 30 000 cSt by the | | | | |
| company Dow Corning | | | | |
| Guar gum modified with | 0.1 g | 0.16 g | 0.1 g | 0.05 g |
| 2,3- | | | | |
| epoxypropyltrimethyl- | | | | |
| ammonium chloride, | | | | |
| sold under the name | | | | |
| Jaguar C13S by | | | | |
| the company Rhodia | | | | |
| Chimie | | | | |
| 1-(Hexadecyloxy)-2- | 2.5 g | | 2.5 g | |
| octadecanol/cetyl alcohol | | | | |
| mixture | | | | |
| Behenyl alcohol | | 1.5 g | | 1.5 g |
| Distearyl ether | | 1.5 g | | 1.5 g |
| Preserving agents, | qs | qs | qs | qs |
| fragrance | | | | |
| pH agents, qs | pH 7.0 | pH 7.5 | pH 5.3 | pH 6.5 |
| Demineralized waterqs | 100.0 g | 100.0 g | 100.0 g | 100.0 g |

The compositions are stable at least one week at an ambient temperature (about 20-25° C.). Moistened hair is not laden and is easy to shape.

What is claimed is:

1. A cosmetic composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymers whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

2. The composition according to claim 1, characterized in that, in the at least one copolymer, the methacrylic acid residue is in an amount ranging from 20% to 80% by weight relative to the total weight of the copolymer.

3. The composition according to claim 2, characterized in that, in the at least one copolymer, the methacrylic acid residue is in an amount ranging from 25% to 75% by weight relative to the total weight of the copolymer.

4. The composition according to claim 3, characterized in that, in the at least one copolymer, the methacrylic acid residue is in an amount ranging from 35% to 60% by weight relative to the total weight of the copolymer.

5. The composition according to claim 1, characterized in that, in the at least one copolymer, the alkyl acrylate residue is in an amount ranging from 15% to 80% by weight relative to the total weight of the copolymer.

6. The composition according to claim 5, characterized in that, in the at least one copolymer, the alkyl acrylate residue is in an amount ranging from 25% to 75% by weight relative to the total weight of the copolymer.

7. The composition according to claim 6, characterized in that, in the at least one copolymer, the alkyl acrylate residue is in an amount ranging from 40% to 65% by weight relative to the total weight of the copolymer.

8. The composition according to claim 1, characterized in that, in the at least one copolymer, the alkyl acrylate residue is chosen from methyl acrylate, ethyl acrylate, and butyl acrylate residues.

9. The composition according to claim 8, characterized in that the alkyl acrylate residue is ethyl acrylate residue.

10. The composition according to claim 1, wherein the at least one copolymer chosen from crosslinked and non-crosslinked copolymers of methacrylic acid and of a C₁-C₄ alkyl acrylate is crosslinked.

11. The composition according to claim 1, wherein the at least one copolymer chosen from crosslinked and non-crosslinked copolymers of methacrylic acid and of a C₁-C₄ alkyl acrylate is partially or totally crosslinked with at least one crosslinking agent.

12. The composition according to claim 11, wherein the at least one crosslinking agent is an ethylenically polyunsaturated crosslinking agent.

13. The composition according to claim 11, characterized in that the content of the at least one crosslinking agent ranges from 0.01% to 5% by weight relative to the total weight of the copolymer.

14. The composition according to claim 13, characterized in that the content of the at least one crosslinking agent ranges from 0.03% to 3% by weight relative to the total weight of the copolymer.

15. The composition according to claim 14, characterized in that the content of the at least one crosslinking agent ranges from 0.05% to 1% by weight relative to the total weight of the copolymer.

16. The composition according to claim 1, characterized in that the at least one copolymer chosen from crosslinked and non-crosslinked copolymers of methacrylic acid and of a C₁-C₄ alkyl acrylate is in the form of a dispersion of particles in water.

17. The composition according to claim 16, characterized in that the number-average size of the particles of the copolymer in the dispersion ranges from 10 to 500 nm.

18. The composition according to claim 17, characterized in that the number-average size of the particles of the copolymer in the dispersion ranges from 20 to 200 nm.

19. The composition according to claim 18, characterized in that the number-average size of the particles of the copolymer in the dispersion ranges from 50 to 150 nm.

20. The composition according to claim 1, characterized in that the at least one copolymer chosen from crosslinked and non-crosslinked copolymers of methacrylic acid and of a C₁-C₄ alkyl acrylate is the crosslinked copolymer of methacrylic acid and of an ethyl acrylate in the form of an aqueous dispersion at 30% by weight.

21. The composition according to claim 1, characterized in that the polydialkylsiloxanes are chosen from polydimethylsiloxanes comprising trimethylsilyl end groups.

22. The composition according to claim 1, characterized in that the polydialkylsiloxanes have a viscosity, measured at 25° C., ranging from 0.2 to 2.5 m²/s.

23. The composition according to claim 1, characterized in that the nacreous and opacifying agents are chosen from:
i) esters of polyols comprising at least two carbon atoms and of C₁₀-C₃₀ fatty acids;
ii) C₁₀-C₃₀ fatty acid alkanolamides;
iii) esters of long-chain monoalcohols and of C₁₀-C₃₀ fatty acids;
iv) C₁₀-C₃₀ fatty alkyl ethers;
v) long-chain esters of C₁₀-C₃₀ alkanolamides;
vi) single-chain fatty alcohols comprising at least 20 carbon atoms;
vii) C₁₀-C₃₀ amine oxides;
viii) N,N-dihydrocarbyl(C₁₀-C₃₀)amidobenzoic acids and their salts;
ix) alcohols comprising from 27 to 48 carbon atoms and comprising one or two groups chosen from ether, thioether and sulphoxide groups; and
x) titanium oxides and micas.

24. The composition according to claim 23, characterized in that the opacifying and nacreous agents are chosen from ethylene glycol monostearate, ethylene glycol distearate, distearyl ether, behenyl alcohol and 1-(hexadecyloxy)-2-octadecanol.

25. The composition according to claim 1, characterized in that the cationic polymers have a cationic density ranging from 0.05 to 1 meq/g.

26. The composition according to claim 1, characterized in that the cationic polymers are chosen from those comprising at least one group chosen from primary, secondary, tertiary and quaternary amine groups that may either form part of the main polymer chain, or be borne by a side substituent that is directly attached to the main polymer chain.

27. The composition according to claim 1, characterized in that the cationic polymers are chosen from:
(1) homopolymers and copolymers derived from acrylic or methacrylic esters or amides and comprising at least one of the units of the following formulae: 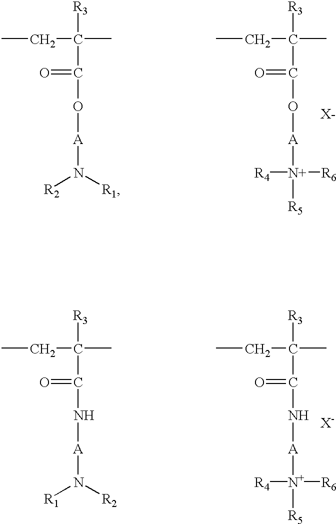
wherein:
R₃, which may be identical or different, is chosen from a hydrogen atom and a CH₃ radical;
A, which may be identical or different, is chosen from linear and branched alkyl groups of 1 to 6 carbon atoms, and hydroxyalkyl groups of 1 to 4 carbon atoms;
R₄, R₅ and R₆, which may be identical or different, are chosen from alkyl groups comprising from 1 to 18 carbon atoms and benzyl radicals;
R₁ and R₂, which may be identical or different, are chosen from hydrogen and alkyl groups comprising from 1 to 6 carbon atoms;
X⁻ is an anion chosen from anions derived from an acid chosen from inorganic and organic acids;
(2) cationic polysaccharides;
(3) polymers comprising at least one piperazinyl unit and at least one radical chosen from divalent alkylene and hydroxyalkylene radicals comprising at least one chain chosen from straight and branched chains, optionally interrupted by at least one entity chosen from oxygen, sulphur and nitrogen atoms, and aromatic and heterocyclic rings, and the oxidation and quaternization products of the polymers;
(4) water-soluble polyamino amides prepared by polycondensation of an acidic compound with a polyamine; optionally the polyamino amides being crosslinked with a crosslinking agent chosen from an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide and an oligomer resulting from the reaction of a difunctional compound reactive with a compound chosen from a bis-halohydrin, a bis-azetidinium, a bis-haloacyldiamine, a bis-alkyl halide, an epihalohydrin, a diepoxide and a bis-unsaturated derivative, wherein the crosslinking agent is in an amount ranging from 0.025 to 0.35 mol per amine group of the polyamino amides; further optionally, these polyamino amides being alkylated; even further optionally, if the polyamino amides comprise at least one tertiary amine function, the polyamino amides can be quaternized;
(5) polyamino amide derivatives resulting from the condensation of polyalkylene polyamines with polycarboxylic acids followed by an alkylation with difunctional agents;
(6) polymers obtained by reaction of a polyalkylene polyamine comprising two primary amine groups and at least one secondary amine group with a dicarboxylic acid chosen from diglycolic acids and saturated aliphatic dicarboxylic acids comprising from 3 to 8 carbon atoms;
(7) cyclopolymers chosen from cyclopolymers of alkyldiallylamine and cyclopolymers of dialkyldiallylammonium;
(8) quaternary diammonium polymers comprising repeating units corresponding to the formula: 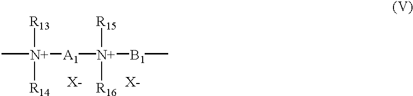
wherein:
R₁₃, R₁₄, R₁₅ and R₁₆, which may be identical or different, are chosen from aliphatic, alicyclic and arylaliphatic radicals comprising from 1 to 20 carbon atoms and lower hydroxyalkylaliphatic radicals, or R₁₃, R₁₄, R₁₅ and R₁₆, together or separately, constitute, with the nitrogen atoms to which they are attached, heterocycles optionally comprising a second hetero atom other than nitrogen, or R₁₃, R₁₄, R₁₅ and R₁₆ are chosen from linear and branched C₁-C₆ alkyl radicals substituted with a group chosen from nitrile, ester, acyl and amide groups and groups of —CO—O—R₁₇—D and —CO—NH—R₁₇—D, wherein R₁₇ is chosen from alkylene groups and D is chosen from quaternary ammonium groups;
A₁ and B₁, which may be identical or different, are chosen from linear and branched, saturated and unsaturated polymethylene groups comprising from 2 to 20 carbon atoms, and which may comprise, linked to or intercalated in the main chain, at least one entity chosen from aromatic rings, oxygen and sulphur atoms, and sulphoxide, sulphone, disulphide, amino, alkylamino, hydroxyl, quaternary ammonium, ureido, amide and ester groups; and
X⁻ is an anion chosen from anions derived from an acid chosen from inorganic and organic acids;
optionally A₁, R₁₃ and R₁₅ form, with the two nitrogen atoms to which they are attached, a piperazine ring; if A₁ is chosen from linear and branched, saturated and unsaturated alkylene and hydroxyalkylene radicals, B₁ can also be chosen from groups (CH₂)ₙ—CO—D—OC—(CH₂)ₙ—, wherein n ranges from 1 to 100, and D is chosen from:
a) glycol residues of formula: —O—Z—O—, where Z is chosen from linear and branched hydrocarbon-based radicals and groups corresponding to one of the following formulae:
—(CH₂—CH₂—O)ₓ—CH₂—CH₂— and
[CH₂—CH(CH₃)—O]_{y}—CH₂—CH(CH₃)—
wherein x and y, which may be identical or different, are chosen from integers ranging from 1 to 4, representing a defined and unique degree of polymerization or any number ranging from 1 to 4 representing an average degree of polymerization;
b) bis-secondary diamine residues;
c) bis-primary diamine residues of formula : —NH—Y—NH—, wherein Y is chosen from linear and branched hydrocarbons radicals, and the divalent radical
—CH₂—CH₂—S—S—CH₂—CH₂—; and
d) a ureylene group of formula: —NH—CO—NH—;
(9) polyquaternary ammonium polymers comprising units of formula (VI): 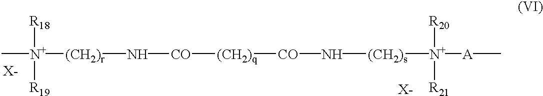
wherein:
R₁₈, R₁₉, R₂₀ and R₂₁, which may be identical or different, are chosen from a hydrogen atom and methyl, ethyl, propyl, β-hydroxyethyl, β-hydroxypropyl and —CH₂CH₂(OCH₂CH₂)ₚOH radicals,
wherein p is equal to 0 or is chosen from integers ranging from 1 to 6, with the proviso that R₁₈, R₁₉, R₂₀ and R₂₁ do not simultaneously represent a hydrogen atom,
r and s, which may be identical or different, are chosen from integers ranging from 1 to 6,
q is equal to 0 or chosen from integers ranging from 1 to 34,
X⁻ is a halogen anion,
A is chosen from divalent radicals;
(10) quaternary polymers of vinylpyrrolidone and of vinylimidazole,
(11) polyamines, under the reference name “Polyethylene glycol (15) tallow polyamine” in the CTFA dictionary.
(12) crosslinked polymers of methacryloyloxy(C₁-C₄)alkyltri(C₁-C₄)alkylammonium salts; and
(13) polyalkyleneimines, polymers comprising at least one unit chosen from vinylpyridine units and vinylpyridinium units, condensates of polyamines and of epichlorohydrin, quaternary polyureylenes and chitin derivatives.

28. The composition according to claim 27, wherein in (8)(b) the bis-secondary diamine residues are chosen from piperazine derivatives.

29. The composition according to claim 27, wherein A in (9) is —CH₂—CH₂—O—CH₂—CH₂—.

30. The composition according to claim 27, wherein in (13) the polyalkyleneimines are chosen from polyethyleneimines.

31. The composition according to claim 1, characterized in that the cationic polymers are chosen from cationic cyclocopolymers, cationic polysaccharides, quaternary polymers of vinylpyrrolidone and of vinylimidazole, and crosslinked homopolymers and copolymers of methacryloyloxy(C₁-C₄)alkyltri(C₁-C₄)alkylammonium salts.

32. The composition according to claim 31, characterized in that the cationic polysaccharides are chosen from guar gums modified with a 2,3-epoxypropyltrimethylammonium salt.

33. The composition according to claim 31, characterized in that the cationic polysaccharides are chosen from hydroxyethylcelluloses that have reacted with an epoxide substituted with a trimethylammonium group.

34. The composition according to claim 1, characterized in that the amphoteric polymers are chosen from:
(1) polymers resulting from the copolymerization of at least one monomer derived from a vinyl compound bearing a carboxylic group and at least one basic monomer derived from a substituted vinyl compound comprising at least one basic atom, optionally the substituted vinyl compound is a dialkyldiallylammonium salt;
(2) polymers comprising at least one unit derived from:
a) at least one monomer chosen from acrylamides and methacrylamides substituted on the nitrogen with an alkyl radical,
b) at least one acidic comonomer comprising at least one reactive carboxylic group, and
c) at least one basic comonomer;
(3) crosslinked and alkylated polyamino amides partially or totally derived from polyamino amides of general formula: 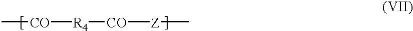
in which R₄ is chosen from divalent radicals derived from a saturated dicarboxylic acid, a mono- and dicarboxylic aliphatic acid comprising an ethylenic double bond, an ester of a lower alkanol comprising 1 to 6 carbon atoms, of these acids and radicals derived from the addition of any one of the acids to an amine chosen from bis(primary) and bis(secondary) amines, and Z is chosen from bis(primary), mono- and bis(secondary) polyalkylene-polyamine radicals; the polyamino amides being crosslinked by addition of a difunctional crosslinking agent chosen from epihalohydrins, diepoxides, dianhydrides and bis-unsaturated derivatives, using from 0.025 to 0.35 mol of the crosslinking agent per amine group of the polyamino amides and alkylated by the action of a reactant chosen from acrylic acids, chloroacetic acids, alkane sultones, and salts thereof;
(4) polymers comprising at least one zwitterionic unit of formula (IX): 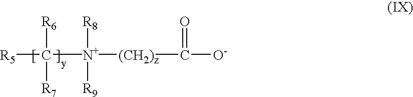
in which R₅ is chosen from polymerizable unsaturated groups,
y and z, which may be identical or different, are chosen from integers ranging from 1 to 3,
R₆ and R₇, which may be identical or different, are chosen from a hydrogen atom, methyl, ethyl and propyl groups,
R₈ and R₉, which may be identical or different, are chosen from a hydrogen atom and alkyl radicals such that the sum of the carbon atoms in R₈ and R₉ does not exceed 10;
(5) polymers derived from chitosan comprising at least one monomer unit corresponding to formulae (X), (XI) and (XII) below: 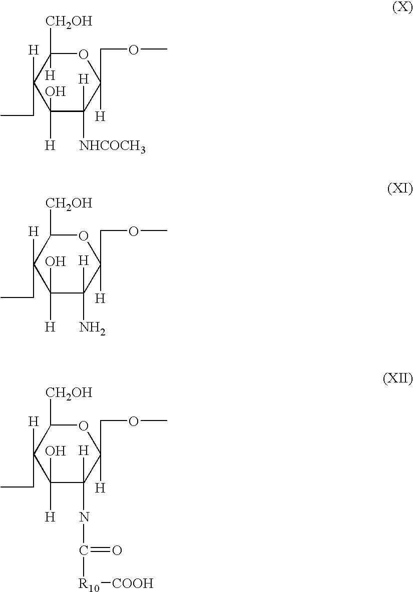
the unit (X) being in an amount ranging from 0 to 30%, the unit (XI) in an amount ranging from 5% to 50% and the unit (XII) in an amount ranging from 30% to 90%, wherein in the unit (XII), R₁₀ is chosen from radicals of formula: 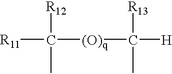
wherein q is equal to 0 or 1,
if q=0, R₁₁, R₁₂ and R₁₃, which may be identical or different, are chosen from a hydrogen atom, methyl, hydroxyl, acetoxy and amino residues, monoalkylamine residues and dialkylamine residues which are optionally interrupted by at least one nitrogen atom and/or optionally substituted with at least one group chosen from amine, hydroxyl, carboxyl, alkylthio and sulphonic group, alkylthio residues in which the alkyl group bears an amino residue, at least one of the radicals R₁₁, R₁₂ and R₁₃ being, in this case, a hydrogen atom;
or, if q=1, R₁₁, R₁₂ and R₁₃, which may be identical or different, are chosen from a hydrogen atom, and the salts formed by these compounds with bases or acids;
(6) polymers derived from the N-carboxyalkylation of chitosan; (7) polymers corresponding to the general formula (XIII): 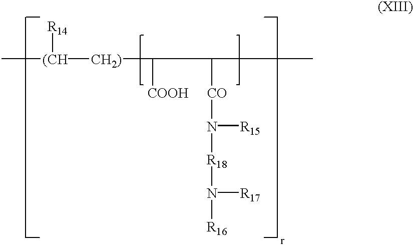
wherein R₁₄ is chosen from a hydrogen atom, CH₃O, CH₃CH₂O and phenyl radicals,
R₁₅ is chosen from hydrogen and lower alkyl radicals, R₁₆ is chosen from hydrogen and lower alkyl radicals, R₁₇ is chosen from lower alkyl radicals and radicals corresponding to the formula: —R₁₈—N(R₁₆)₂, wherein R₁₈ is chosen from —CH₂—CH₂—, —CH₂—CH₂—CH₂— and —CH₂—CH(CH₃)— groups, R₁₆ is chosen from hydrogen and lower alkyl radicals, and the higher homologues of these radicals comprising up to 6 carbon atoms, r is chosen such that the number-average molecular weight of the polymer ranges from 500 to 6,000,000;
(8) amphoteric polymers of the type —D—X—D—X— chosen from:
a) polymers obtained by the action of chloroacetic acid or sodium chloroacetate on compounds comprising at least one unit of formula:
—D—X—D—X—D— (XIV)
wherein D is a radical 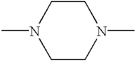
and X is chosen from the symbols of E and E′, wherein E and E′, which may be identical or different, are chosen from divalent alkylene radicals comprising at least one chain chosen from straight and branched chains comprising up to 7 carbon atoms in the main chain, wherein the main chain is optionally substituted with at least one hydroxyl group and E or E′ optionally comprises at least one atom chosen from oxygen, nitrogen and sulphur atoms, and 1 to 3 rings chosen from aromatic and heterocyclic rings; wherein the oxygen, nitrogen and sulphur atoms are present in the form of at least one group chosen from ether, thioether, sulphoxide, sulphone, sulphonium, alkylamine, alkenylamine, hydroxyl, benzylamine, amine oxide, quaternary ammonium, amide, imide, alcohol, ester and urethane groups;
b) polymers of formula:
—D—X—D—X— (XV)
wherein D is a radical 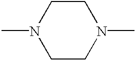
and X is chosen from the symbols E and E′ and wherein at least one X is chosen from E′ wherein E is chosen from divalent alkylene radicals comprising at least one chain chosen from straight and branched chains comprising up to 7 carbon atoms in the main chain, wherein the main chain is optionally substituted with at least one hydroxyl group and E optionally comprises at least one atom chosen from oxygen, nitrogen and sulphur atoms, and 1 to 3 rings chosen from aromatic and heterocyclic rings; wherein the oxygen, nitrogen and sulphur atoms are present in the form of at least one group chosen from ether, thioether, sulphoxide, sulphone, sulphonium, alkylamine, alkenylamine, hydroxyl, benzylamine, amine oxide, quaternary ammonium, amide, imide, alcohol, ester and urethane groups;
and E′ is chosen from divalent alkylene radicals comprising at least one chain chosen from straight and branched chains comprising up to 7 carbon atoms in the main chain, wherein the main chain is optionally substituted with at least one hydroxyl radical and the divalent alkylene radicals comprise at least one nitrogen atom, the at least one nitrogen atom being substituted with an alkyl chain, wherein the alkyl chain is optionally interrupted by an oxygen atom and wherein the alkyl chain comprises at least one functional group chosen from carboxyl functional groups and hydroxyl functional groups and wherein the alkyl chain is betainized by reaction with a reactant chosen from chloroacetic acids and sodium chloroacetate; and
(9) (C₁-C₅)alkyl vinyl ether/maleic anhydride copolymers partially modified by semiamidation with an N,N-dialkylaminoalkylamine or by semiesterification with an N,N-dialkanolamine.

35. The composition according to claim 34, wherein in (1) the at least one monomer derived from a vinyl compound bearing a carboxylic group is chosen from acrylic acids, methacrylic acids, maleic acids, and α-chloroacrylic acids.

36. The composition according to claim 34, wherein in (1) the at least one basic monomer derived from a substituted vinyl compound comprising at least one basic atom is chosen from dialkylaminoalkyl methacrylates, dialkylaminoalkyl acrylates, dialkylaminoalkylmethacrylamide and dialkylaminoalkylacrylamide.

37. The composition according to claim 34, wherein in (1) the dialkyldiallylammonium salt is dimethyldiallylammonium chloride.

38. The composition according to claim 34, wherein in (2)(c) the at least one basic comonomer is chosen from esters comprising primary, secondary, tertiary and quaternary amine substituents of acrylic and methacrylic acids and the products of quaternization of dimethylaminoethyl methacrylate with dimethyl or diethyl sulphate.

39. The composition according to claim 34, wherein in (3) Z represents:
a) in an amount ranging from 60 to 100 mol %, the radical of formula (VIII) 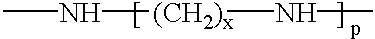
wherein x=2 and p=2 or 3, or x=3 and p=2 the radical being derived from a compound chosen from diethylenetriamine, triethylenetetraamine and dipropylenetriamine;
b) in an amount ranging from 0 to 40 mol %, the radical chosen from the radicals of formula (VIII) in which x=2 and p=1 derived from ethylenediamine, and the radicals derived from piperazine: 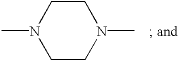
c) in an amount ranging from 0 to 20 mol %, the —NH—(CH₂)₆—NH— radical derived from hexamethylenediamine.

40. The composition according to claim 34, wherein in (4) the polymerizable unsaturated groups are chosen from acrylate, methacrylate, acrylamide and methacrylamide groups.

41. The composition according to claim 34, wherein in (6) the polymers derived from the N-carboxyalkylation of chitosan are chosen from N-carboxymethylchitosans and N-carboxybutylchitosans.

42. The composition according to claim 34, wherein in (7) the lower alkyl radicals are chosen from methyl radicals and ethyl radicals.

43. The composition according to claim 34, wherein in (9) the N,N-dialkylaminoalkylamine is N,N-dimethylaminopropylamine.

44. The composition according to claim 43, characterized in that the amphoteric polymers are chosen from copolymers of dimethyldiallylammonium salt and of acrylic acid.

45. The composition according to claim 1, further comprising at least one additional silicone other than polydialkylsiloxanes comprising trimethylsilyl end groups.

46. The composition according to claim 45, characterized in that the at least one additional silicone is chosen from non-volatile silicones chosen from the family of polyalkylsiloxanes comprising dimethylsilanol end groups and aminosilicones.

47. The composition according to claim 1, further comprising at least one agent that is beneficial for a keratin material, chosen from esters of C₁-C₃₀ carboxylic acids and of C₁-C₃₀ mono- and polyhydroxylated alcohols other than the nacreous and opacifying agents according to claim 23, plant, animal, mineral and synthetic oils, waxes, ceramides and pseudoceramides.

48. The composition according to claim 47, wherein the keratin material is hair.

49. The composition according to claim 1, characterized in that the at least one copolymer chosen from crosslinked and non-crosslinked copolymers of methacrylic acid and of a C₁-C₄ alkyl acrylate is in a concentration ranging from 0.01% to 20% by weight relative to the total weight of the composition.

50. The composition according to claim 49, characterized in that the at least one copolymer chosen from crosslinked and non-crosslinked copolymers of methacrylic acid and of a C₁-C₄ alkyl acrylate is in a concentration ranging from 0.05% to 15% by weight relative to the total weight of the composition.

51. The composition according to claim 1, characterized in that the at least one agent chosen from nacreous and opacifying agents is in an amount ranging from 0.1% to 15% by weight relative to the total weight of the composition.

52. The composition according to claim 51, characterized in that the at least one agent chosen from nacreous and opacifying agents is in an amount ranging from 0.5% to 10% by weight relative to the total weight of the composition.

53. The composition according to claim 52, characterized in that the at least one agent chosen from nacreous and opacifying agents is in an amount ranging from 1% to 5% by weight relative to the total weight of the composition.

54. The composition according to claim 1, characterized in that the at least one polymer chosen from cationic and amphoteric polymers is in a concentration ranging from 0.001% to 20% by weight relative to the total weight of the composition.

55. The composition according to claim 54, characterized in that the at least one polymer chosen from cationic and amphoteric polymers is in a concentration ranging from 0.01% to 10% by weight relative to the total weight of the composition.

56. The composition according to claim 55, characterized in that the at least one polymer chosen from cationic and amphoteric polymers is in a concentration ranging from 0.05% to 1% by weight relative to the total weight of the composition.

57. The composition according to claim 1, characterized in that the at least one silicone chosen from polydialkylsiloxanes comprising trimethylsilyl end groups is in a concentration ranging from 0.001% to 20% by weight relative to the total weight of the composition.

58. The composition according to claim 57, characterized in that the at least one silicone chosen from polydialkylsiloxanes comprising trimethylsilyl end groups is in a concentration ranging from 0.01% to 10% by weight relative to the total weight of the composition.

59. The composition according to claim 45, characterized in that the at least one additional silicone is in a concentration ranging from 0.001% to 20% by weight relative to the total weight of the composition.

60. The composition according to claim 59, characterized in that the at least one additional silicone is in a concentration ranging from 0.01% to 10% by weight relative to the total weight of the composition.

61. The composition according to claim 47, characterized in that the at least one agent that is beneficial for a keratin material is in a concentration ranging from 0.001% and 20% by weight relative to the total weight of the composition.

62. The composition according to claim 61, characterized in that the at least one agent that is beneficial for a keratin material is in a concentration ranging from 0.01% and 10% by weight relative to the total weight of the composition.

63. The composition according to claim 1, further comprising at least one surfactant chosen from anionic, nonionic, amphoteric and cationic surfactants.

64. The composition according to claim 63, characterized in that the at least one surfactant is in a concentration ranging from 0.01% to 50% by weight relative to the total weight of the composition.

65. The composition according to claim 64, characterized in that the at least one surfactant is in a concentration ranging from 0.1% to 40% by weight relative to the total weight of the composition.

66. The composition according to claim 65, characterized in that the at least one surfactant is in a concentration ranging from 0.5% to 30% by weight relative to the total weight of the composition.

67. The composition according to claim 63, characterized in that the at least one surfactant is chosen from anionic surfactants, mixtures of at least one anionic surfactant and at least one amphoteric surfactant, and mixtures of at least one anionic surfactant and at least one nonionic surfactant.

68. The composition according to claim 1, further comprising at least one additive chosen from thickeners, antidandruff agents, anti-seborrhoeic agents, fragrances, hydroxy acids, electrolytes, fatty acid esters, preserving agents, silicone and non-silicone sunscreens, anionic and nonionic polymers, proteins, protein hydrolysates, 18-methyleicosanoic acid, vitamins, provitamins, fluoro and perfluoro oils.

69. The composition according to claim 68, wherein the provitamins are chosen from panthenol.

70. A composition according to claim 1, characterized in that the composition is in the form of a shampoo, a conditioner, a permanent-waving, straightening, dyeing or bleaching composition for hair, a rinse-out composition to be applied between the two steps of a permanent-waving or hair-straightening operation, or a washing composition for a body.

71. A composition for washing or caring for a keratin material comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns, said composition being effective for washing or caring for a keratin material.

72. A method for washing or caring for a keratin material comprising applying to the keratin material a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

73. A process for treating a keratin material comprising applying to the keratin material a cosmetic composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns, and then optionally rinsing it out with water.

74. The process according to claim 73, wherein the keratin material is hair.

75. A composition comprising, in a cosmetically acceptable medium, one composition (1) comprising at least one copolymer chosen from crosslinked and non-crosslinked copolymers of methacrylic acid and of a C₁-C₄ alkyl acrylate and another composition (2) comprising at least one polymer chosen from cationic and amphoteric polymers whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

76. A method of manufacturing a cosmetic composition, comprising including in said composition at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

77. A composition to give hair sheen comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising at trimethylsilyl end groups, the volume-average size of the silicone particles before introduction into the composition and/or in the final composition being greater than or equal to 2 microns, said composition being effective to give hair sheen.

78. A method to give hair sheen comprising applying to hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

79. A composition to give hair lightness comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns, said composition being effective to give hair lightness.

80. A method to give hair lightness comprising applying to hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

81. A composition to give hair softness comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns, said composition being effective to give hair softness.

82. A method to give hair softness comprising applying to hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

83. A composition to give hair a smooth feel comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns, said composition being effective to give hair a smooth feel.

84. A method to give hair a smooth feel, comprising applying to hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.

85. A composition to give hair suppleness comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns, said composition being effective to give hair suppleneess.

86. A method to give hair suppleness comprising applying to hair a composition comprising, in a cosmetically acceptable medium, at least one copolymer chosen from crosslinked and non-crosslinked copolymers of methacrylic acid and of a C₁-C₄ alkyl acrylate, at least one polymer chosen from cationic and amphoteric polymer whose cationic charge density is less than 1 meq/g, at least one agent chosen from nacreous and opacifying agents and at least one silicone chosen from polydialkylsiloxanes comprising trimethylsilyl end groups, the volume-average size of the silicone particles before introduction into the composition and/or in the final composition being greater than or equal to 2 microns.
